(19) United States
(12) Patent Application Publication
(10) Pub. No.: US 2005/0118272 A1
(43) Pub. Date: Jun. 2, 2005

(54) Micronized pharmaceutical or nutraceutical powder with immediate release

(76) Inventors: Jerome Besse, Listrac Medoc (FR); Laurence Besse, Listrac Medoc (FR)

Correspondence Address: JACOBSON HOLMAN PLLC, 400 SEVENTH STREET N.W., SUITE 600, WASHINGTON, DC, 20004, US

(21) Appl. No.: 10/500,213

(22) PCT Filed: Dec. 27, 2002
(86) PCT No.: PCT/FR02/04575

(51) Int. Cl. A61K 38/28; A61L 9/04; A61K 9/14; A61K 31/4965; A61K 31/557

(52) U.S. Cl. 424/489; 514/169; 514/304; 514/255.06; 514/12; 514/3; 514/263.31; 514/263.38; 514/568; 514/49; 514/557; 514/343; 514/305; 514/559; 514/573; 514/269; 514/254.07; 514/509

(57) ABSTRACT

The invention concerns a micronized pharmaceutical or nutraceutical powder with immediate release having a grain size distribution of not more than 100 μm, and comprising the combination of at least an active substance, at least a wetting agent and at least a diluent.

The present invention relates to an immediate-release pharmaceutical or nutraceutical micronized powder for mucosal application, in particular buccal.

The use of a micronized powder according to the invention for preparing a pharmaceutical or nutraceutical composition allows a rapid release (or “flash”) of the active substance when the composition comprising it is administered mucosally, in particular buccal.

Pharmaceutical forms which allow rapid release of an active substance are already known. They are tablets of the “lyoc” type or tablets which disintegrate rapidly in the mouth, such as for example the Zydis® (Scherer®) technology, or film-type systems provided in the form of a “wafer”, i.e. films for buccal application which allow more or less rapid dissolution of the active substances.

This being so, these two pharmaceutical forms have several drawbacks. The tablets suffer from a significant friability, which makes them delicate to handle, and, moreover, their disintegration time is very often longer than 10 seconds. The films are difficult to apply due to their very small thickness. In addition, the two pharmaceutical forms suffer from a major drawback in that they allow only a relatively low load of active substance, diverse and varied excipients being required for their structural integrity.

The Applicant Companies have therefore sought to develop a pharmaceutical form which can overcome the drawbacks encountered by the prior formulations.

They have thus succeeded in developing a powder, the use of which in a pharmaceutical or nutraceutical composition allows rapid and immediate release of the active substance alone or in combination, when said composition is administered buccally.

For the purpose of the present invention, the expression “rapid and immediate release” is intended to mean release of all of the active substance(s) in less than 30 seconds, preferably less than 15 seconds and even more preferentially in less than 10 seconds.

The powder according to the invention, unlike the tablets and films of the prior art, is delicate neither in terms of its handling nor in its application. It also allows a considerable active substance load. Specifically, the load of active substances per dose unit can be considerably greater than the 20 mg imposed in particular by the technology of the films of the “wafer” type or equivalent.

The powder according to the present invention therefore has many advantages compared to the known pharmaceutical forms in the prior art.

Thus, the present invention relates to an immediate-release pharmaceutical or nutraceutical micronized powder having a particle size of at most 100 μm and comprising the combination of at least one active substance, at least one wetting agent, and at least one diluent.

Preferably, the immediate-release micronized powder of the invention comprises, on the basis of the total weight of the composition, from 0.001% to 99% by weight of active substance(s), from 1% to 60% by weight of wetting agent(s) and from 0.1% to 99% of diluent(s). Those skilled in the art adapt the levels of the various constituents of the immediate-release micronized powder in accordance with conventional techniques for preparing pharmaceutical formulations such as for example those described in (i) J. Control Release, 1999, Vol. 61: 175-183, (ii) J. Pharm., 2000, 171-277, (iii) J. Control Release, 2001, Vol. 77: 1-6 or (iv) J. Pharm. Pharmacol., 1996, Vol. 48: 255, so that the powder has the physical, mechanical and chemical features defined in the present disclosure, more particularly the features of particle size, of release kinetic of the active substance(s) or that of residual humidity.

By active substance, it is meant according to the invention any substance having a measurable activity of therapeutical, cosmetic or nutraceutical nature, towards the human or animal body to which this active substance is applied or administered.

By wetting agent, it is meant according to the invention an agent which accelerates the solubilization and/or the dissolution of the active substance(s) and the other excipients contained in the micronized powder. In particular, a wetting agent according to the invention is characterised in that it allows a high wettability index of said micronized powder, as it may be visualized by the measurement of the contact angle (α) with the aid of a goniometer, which is low and is preferably in the range of between 0 and 90°, more preferably between 0 and 60° and most preferably between 0 and 45°.

By diluent, it is meant according to the invention, an agent used in order to complete the composition of the micronized powder containing the active substance(s) until a predetermined total volume containing a selected amount of the active substance(s) is obtained, the volume of the active substance(s) themselves, depending on the nature of these active substances, being in general insufficient for achieving a final micronized powder the desired volume of which comprises the suitable amount of said active substance(s).

According to the invention, it has been shown that a micronized powder having the combination of the above features and possessing a particle size of at most 100 μm, because of a great active area, allowed an excellent bioavailability of the active substance(s) it contains, for the target sites or cell receptors intended on the mucous membrane.

By “particle size” of an immediate-release micronized powder according to the invention, it is meant the mean size of the grains that constitute it. The mean size of the grains can be measured by any conventional technique known per se. More particularly, those skilled in the art can use a measurement with the aid of a laser granulometry device of the Beckman Coulter® or Malvern® type, as described in the examples.

The applicant has noticed that the grain size distribution of the immediate-release micronized powder according to the invention follows a narrow Gauss curve, with the particle size value corresponding therefore to the real size of the most part of the grains contained in said powder.

The immediate-release micronized powder according to the invention conveniently has a residual humidity of between 0.01% and 15%, preferably between 0.1% and 5%, as measured with a humidity analyser type Sartorius® MA 30 sold by the Sartorius Company and used in accordance with the manufacturer recommendations, as illustrated in the examples. The low residual humidity of the immediate-release micronized powder according to the invention allows to avoid, or to say the least to strongly decrease, the aggregate formation between the grains contained in said powder. Indeed, the aggregate formation is likely to affect the active area value of the powder in contact with the mucous membranes upon its application, and as a result the bioavaibility value of the active substance(s) for the target sites or receptors in the mucous membranes.

It has also been shown according to the invention that, within certain limits, the greater the micronized powder particle size is low, the greater the bioavailability of the active substance(s) towards the intended target sites is increased and the greater the time required for the total release of the active substance(s) to the target sites or receptors on the mucous membrane is reduced.

Thus, preferentially, the micronised powder according to the invention has a particle size of at most 50 μm, and most preferably of at most 10 μm.

At the example 1, there is illustrated an immediate-release micronized powder according to the invention having a particle size of less than 3 μm.

It has also been shown according to the invention that with a micronized powder having a particle size of less than 0.01 μm, the immediate release ability of the active substances was altered, more particularly because of a cluster agglomeration of the powder grains with each other. Thus, with a micronized powder having a too fine particle size, the bioavaibility of the active substance(s) for the target sites on the mucous membranes is reduced because of the retention of the active substance(s) within the powder, at the middle of the grains agglomerates which are forming. In other words, unlike that could be expected, a too large reduction of the micronized powder particle size, below 0.01 μm, has the effect of reducing the active area of said powder in the contact with the mucous membranes, in comparison with a micronized powder with a greater particle size, for example from 1 μm to 5 μm.

According to a preferred ambodiment of the immediate-release micronized powder of the invention, said powder presents a particle size of between 0.01 μm and 100 μm, advantageously between 0.1 μm and 100 μm, more preferably between 1 μm and 50 μm and most preferably between 1 μm and 20 μm.

The immediate-release micronized powder according to the invention has a dissolution kinetic in an aqueous medium of less than thirty seconds, and most often of less than ten seconds, whether in buffers having a pH of from 5 to 9, or in an aqueous solution of artificial saliva.

Thus, according to an advantageous feature of the immediate-release micronized powder of the invention, said powder allows the release of all of the active substance(s) in less than 30 seconds, advantageously in less than 15 seconds, and most preferably in less than 10 seconds.

The immediate-release micronized powder of the invention is specifically suitable for the rapid release of an active substance, or a combination of active substances, in situ, at the mucous membranes, particularly buccal mucosa.

According to a preferred embodiment of the immediate-release micronized powder, the active substance(s) themeselves are in micronized form.

Thus, according to a more preferred embodiment of the micronized powder of the invention, the active substances are micronized with the other ingredients. This further increases the ability of the powder to release rapidly and homogenously the active substance(s) because of an increase of the contact area thereof with the mucous membrane. Furthermore, several packaging systems of the powder are particularly well suited such as the spray of micronized products or the use of single-dose packets or thermally moulded capsules provided with a peelable operculum.

The active substances of the powder used according to the invention may be selected from those conventionally used in the following pharmacotherapeutic families: allergology, anaesthesia/reanimation, cancerology and haematology, cardiology and angiology, contraception and interruption of pregnancy, dermatology, endocrinology, gastroenterohepatology, gynaecology and obstetrics, immunology and transplantation drug, infectiology and parasitology, metabolism diabetes and nutrition, neurology/psychiatry, ophthalmology, otorhinolaryngology, pneumology, rheumatology, stomatology, toxicology, urology/nephrology, and also from analgesics/antipyretic and antispasmodics, anti-inflammatory agents, contrast products used in radiology, haemostatics, and blood treatment products and derivatives.

Advantageously, the active substances may be selected from the group consisting of the active substances which cross the mucosal barrier and reach the systemic circulation, such as the non limiting examples given hereinafter: cyproterone acetate, norethisterone acetate, progesterone, 3-keto-desogestrel, norgestimate, laevonorgestrel, desogestrel, gestodene, natural estrogens such as estradiol and derivatives thereof, synthetic estrogens such as ethinylestradiol, Δ-4-androstenedione, testosterone, dihydrotestosterone or androstanolone, DHEA, trinitrine, fentanyl, nitroglycerine, nicotine (nicotine S(−)), scopolamine, clonidine, isosorbide dinitrate, alclometasone dipropionate, phloroglucinol, molsidomine, and combinations thereof.

They may also be selected from the active substances which cross the mucosal barrier and have a localized action, such as: acetazolamide, acyclovir, adapalene, alclomethasone dipropionate, amcinonide, ameline, bamethan sulphate+escin, betamethasone valerate, betamethasone dipropionate, bufexamac, caffeine, calcipotriol monohydrate, cetrimonium bromide, clobetasol propionate, crilanomer, desonide, dexpanthenol, diclofenac, diflucortolone, valerate, difluprednate, diphenydramine hydrochloride, econazole nitrate, erythromicin, flumetasone pivalate, fluocinolon acetonide, fluocinodine, fluocortolone, fluocortolone hexanoate, fluocortolone pivalate, hydrocortisone, hydrocortisone acetate, ibacitabine, ibuprofen, imiquimod, ketoconazole, ketoprofen, lidocaine, metronidazole, miconazole nitrate, minoxidil, nifluminic acid, penciclovir, benzoyl peroxide, piroxam, iodinated povidone, promestriene, pyrazinobutazone, roxithromycin, sulphacetamide, triamcinolone, tazarotene, tretinoin and isotretinoin, triclocarban, vidarabine monophosphate and combinations thereof.

They may also be selected from the following active substances: β-3-adrenergic agonist, growth hormone, oxybutinin, buprenorphine, pergolide, nestorone, 7α-methyl-19-nortesterone, mecamylamine, salbutamol, clenbuterol, selegiline, buspirone, ketotifen, lidocaine, ketorolac, eptazocine, insulin, α-interferon, prostaglandins, 5-aminolevulinic acid, benzodiazepine alprozolam, diclofenac, fenoprofen, flubiprofen, ketoprofen, methyl phenidate, miconazole, piroxicam, bruprenorphine, alerozolam, dexmedetomidine, prazosin (α-adrenergic antagonist), alprostadil, tulobuterol (β-adrenergic agonist), ethinyl oestradiol+norelgestromin, ketorolac, physostigmine, medindolol (α-adrenergic agonist), rotigotine (dopamine D2 antagonist), thiatolserine and combinations thereof.

They may also be selected from the following active substances: Esomeprazole, Melagatran (in the case of thrombosis), Rosuvastatin, Ezetimide, Pitavastatin (hyperlipidaemia), Mitiglinide (type II diabetes), Cilomilast, Viozan (asthma), Aripipazole (psychiatry), Omapatrilat (hypertensive), Orzel (cancerology), Caspofongin acetate, Voriconazole (infections), new COX inhibitors such as Etoricoxib (inflammation), Valdecoxib (arthritis) and Parecoxib, Substance P antagonist (depression), Darifenacin (urology), Eletriptan (migraine), Alosetron, Tegaserod, Capravirine (HIV), Finasteride (5-alpha reductase inhibitor) and combinations thereof (non-limiting list).

The powder used according to the invention may contain one or more active substances in combination with one another.

For nutraceutical applications, the active substance may be chosen from the list of raw materials authorized as food supplements, such as, for example, from the group consisting of vitamins, mineral salts, brewer's yeast, etc.

The wetting agent may be one conventionally referred to as such, for example, in the european pharmacopoeia or in the United States Pharmacopoeia (USP) in force or any other wetting agents of pharmaceutical or nutraceutical grade. Wetting agent contained in a micronized powder of the invention includes also agents classified in the european pharmacopoeia or in the United States Pharmacopoeia (USP) as surfactants. Indeed, according to a particular aspect of the immediate-release micronized powder of the invention, the surfactants are also used as wetting agents.

Preferably, the wetting agent is selected from the group consisting of polyols such as sorbitol, or glycerin, PEG, hexylene glycol, triacetin, hydrogenated vegetable oils such as hydrogenated castor oil, polyoxy(ethylene)polyoxy(propylene)copolymer such as Lutrol® F68, polyoxyethylene alkyl ethers such as Cremophor®, and the mixtures thereof (non-limiting list).

Preferably, the diluent is selected from the group consisting of calcium or sodium carbonate or bicarbonate, sucrose, mannitol, xylitol, sorbitol, lactose, maltotol, glucose, cellulose or microcrystalline cellulose powder, starch and its derivatives, dibasic calcium phosphate, tribasic calcium phosphate, calcium sulphate, dextrates, dextrins, dextrose excipients, fructose, kaolin, lactitol and mixtures thereof (non-limiting list).

More preferably, the micronized powder according to the invention comprises also at least an antistatic agent.

It was indeed shown according to the invention that the addition of at least an antistatic agent allows to increase significantly the ability of the micronized powder according to the invention to release rapidly all of the active substance(s) that said powder contains. The addition of at least an antistatic agent allows to avoid, or to say the least strongly decreases, the formation of powder aggregates which are due to the low particle size thereof. Thus, the addition of at least an antistatic agent allows to obtain a low particle size micronized powder comprising non aggregates between grains, and the grains of which, well separated from each other, allow to obtain a maximum contact area of the powder with the mucous membranes upon its application on the latter, and as a result, a maximum accessibility or bioavailability of the active substance(s) for corresponding target site or receptors on the mucous membranes.

Preferably, the immediate-release micronized powder of the invention comprises, on the basis of the total weight of the composition, from 0.01% to 10% of one or more antistatic agent(s).

Preferably, an antistatic agent is selected from the group consisting of colloidal silica, magnesium silicate, talc, calcium silicate and tribasic calcium phosphate (non-limiting list).

The powder used according to the invention may also comprise a binding agent selected from the group consisting of acacia, alginic acid, sodium carboxymethylcellulose, microcrystalline cellulose, dextrins, ethylcellulose, gelatin, glucose, guar gum, hydroxypropylmethylcellulose, methylcellulose, poly-ethylene oxide, povidone, pregelatinized starch, and mixtures thereof (non-limiting list).

The powder used according to the invention may also comprise, if necessary, a penetration enhancer, more preferably referred to as “absorption enhancer” in the present disclosure. By “absorption enhancer”, it is meant any molecule promoting the diffusion of an active substance through the skin or the mucous membrane reversibly, and any solubilizing agent or wetting agent promoting the partition of the active substance between the carrier and the corneum of the epiderm or the mucous membrane.

In the cases that the absorption enhancer is also a wetting agent such as defined above, said absorption enhancer is added to the micronized powder composition which already comprises a wetting agent.

The absorption enhancer may be selected from the group consisting of aliphatic fatty acid esters such as isopropyl myristate, fatty acids such as oleic acid; alcohols or polyols, such as ethanol, propylene glycol and polyethylene glycol; the components of essential oils and terpen derivatives (such as eugenol, geraniol, nerol, eucalyptol or menthol); surfactants preferably nonionic, such as polyoxyethylene sorbitan (fatty acid ester), polyoxyethylene alkyl ether, polyoxyethylene derived from castor oil; moisturizers such as glycerin, urea; keratolytic agents, such as alpha-hydroxy acids (lactic acid, citric acid, etc), 23-lauryl ether, aprotinin, azone, benzalkonium chloride, cetylpyridinium chloride, cetyltrimethylammonium bromide, cyclodextrins, dextran sulphate, lauric acid, lysophosphatidylcholine, menthol, methoxysalicylate, methyl oleate, oleic acid, phosphatidylcholine, polyoxyethylene, polysorbate 80, sodium EDTA, sodium glycocholate, sodium glycodeoxycholate, sodium lauryl sulphate, sodium salicylate, sodium taurocholate, sodium taurodeoxycholate, sulphoxides, alkyl glycosides and mixture thereof (non-limiting list). Furthermore, in order to improve the patient compliance, a sweetener and/or a flavorants may be optionally added to the composition.

The sweetener may be selected from the group consisting of aspartam, dextrates, dextrose, fructose, mannitol, sodium or calcium saccharinate, sorbitol, sucralose, sucrose, and mixtures thereof (non limiting list).

The flavorant may be selected from the group consisting of flavors of synthetic, semi-synthetic or natural origin. As an example, mention may be made of mint, pepermint, lemon, banana, strawberry, raspberry, mandarin, orange, vanilla, passion fruit, caramel, and mixtures thereof.

The composition containing the powder used according to the invention is administered mucosally. It may be applied, for example, on the buccal mucousa, the nasal mucousa or the vaginal mucousa, and also sublingually.

Generally, the immediate-release micronized powder of the invention may be used with or in any device allowing its application to the surface of a mucous membrane.

Advantageously, the composition comprising the powder used according to the invention is in a dry form packaged in a spray or in a 4-seal single-dose packet or a 3-seal single-dose packet such as the “stick pack” which is a tubular packet with a longitudinal seal and a seal on each end of the tube, or in a thermally moulded capsule provided with a peelable operculum or in any other packaging suitable for powder administration known to those skilled in the art. These packagings allow a precise dose of active material to be delivered easily.

All the methods known to those skilled in the art may be used in the context of producing the powder used according to the invention.

As an example of a method for preparing a powder, mention may be made of: wet or dry granulation, followed by micronization.

Alternatively, according to another embodiment, the active substance is micronized and then mixed with the excipients in the form of powder, and the mixture thus obtained is granulated, by wet or dry granulation, and then micronized.

Advantageously, to prepare a immediate-release micronized powder according to the invention, (i) the active substance(s), (ii) the wetting agent(s), (iii) the diluent(s), preferentially (iv) the antistatic agent(s) and also optionally (v) the other excipients, such as the binding agent(s) and/or the absorption enhancer(s), are mixed in a mixer-granulator-dryer type device, until the mixture is homogenized. Then, a wetting solution or suspension is incorporated with stirring in order to obtain a wet granule, which is then dryed in order to evaporate the granulation solvent.

The powder is subsequently micronized after calibration.

For the micronization, the conventional air jet method is preferably used, for example by using an air jet micronization equipment type ALPINE or JET MILL, in accordance with the manufacturer recommendations.

The preferred parameters for a micronization on a micronizer GALETTE Alpine 200AS are the following:
Injector: 7 to 8 bars;
Crown: 4 to 6 bars; and
Speed: 25 kg/h.

In a particular test performed by the Applicant, the powder before micronization had a grain mean size (particle size) of about 160 μm. After micronization, the resulting immediate-release micronized powder had a particle size of 2.3 μm.

The active substance on its own or the final mixture of ingredients may be micronised.

The invention is further illustrated, but not limited to, the following figure and examples.

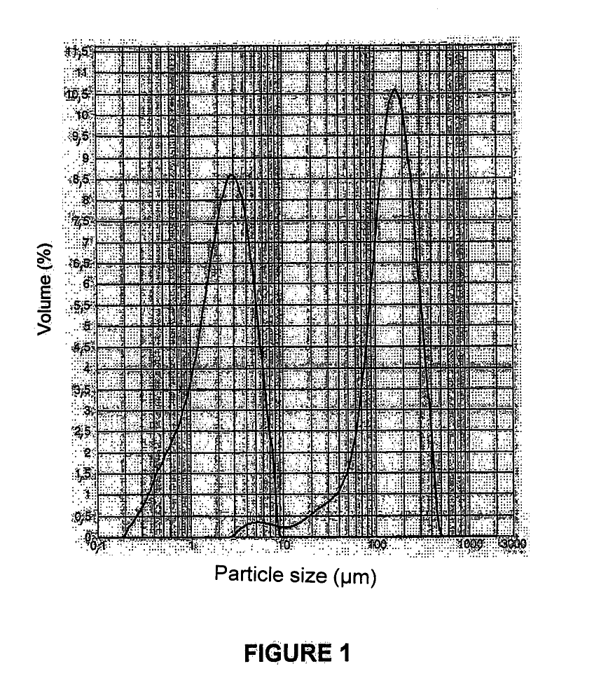

FIG. 1 illustrates the grain size distribution profile of the immediate-release micronized powder of the invention prepared in the example 2, before and after micronization.
In abscissa: Particle size given in μm.
In ordinate: Volume, given as a percentage.

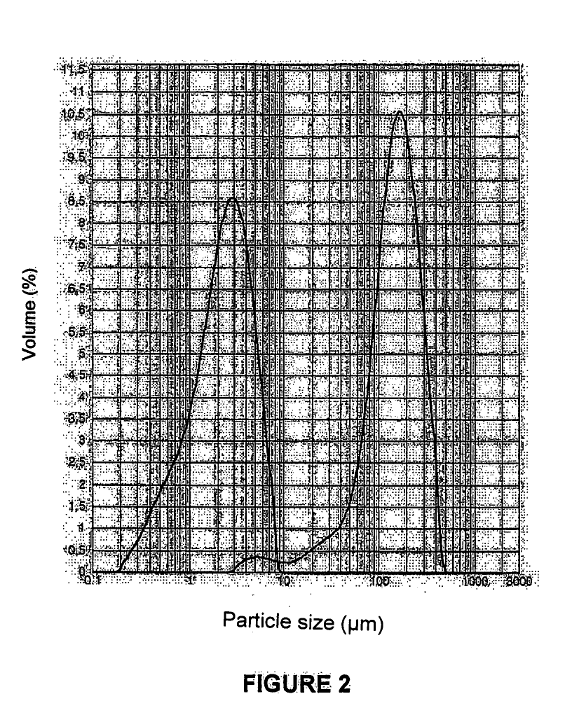

FIG. 2 illustrates the grain size distribution profile of the immediate-release micronized powder of the invention prepared in the example 3, before and after micronization.
In abscissa: Particle size given in μm.
In ordinate: Volume, given as a percentage.

### EXAMPLE 1

### Powders to be Used According to the Invention

Four powders each having the following weight composition are prepared:

TABLE 1

| Composition | Quantity in % |
|---|---|
| Phloroglucinol | 10 |
| Sorbitol | 89 |
| Propylene glycol | 1 |

TABLE 2

| Composition | Quantity in % |
|---|---|
| Testosterone | 10 |
| Sorbitol | 88 |
| Cremophor RH40 | 2 |

TABLE 3

| Composition | Quantity in % |
|---|---|
| Dihydrotestosterone | 5 |
| Xylitol | 90 |
| Glycerol | 3 |
| Tween 80 | 2 |

TABLE 4

| Composition | Quantity in % |
|---|---|
| Molsidomine | 10 |
| Xylitol | 83 |
| Propylene glycol | 5 |
| Montanox 80 | 2 |

The various pulverulent components with the exception of the antistatic agent are mixed in a mixer/granulator of the ROTOLAB ZANCHETTA® mixer/granulator/drier under vacuum type, or equivalent, until the mixture is homogenized. A wetting solution or suspension comprising the liquid component(s) is then incorporated with stirring in order to obtain a wet granule.

This granule is then dried under suitable conditions in order to evaporate the granulation solvent. This granule is then dried and calibrated, and then micronized using an air jet micronization machine of the ALPINE or JETMIL type (or equivalent).

### EXAMPLE 2

### Immediate-Release Powder According to the Invention

A powder having the following composition by weight is prepared.

TABLE 5

| Composition | Quantity in % |
|---|---|
| Apomorphine | 10 |
| Sorbitol | 89.01 |
| Propylene glycol | 0.90 |
| Colloidal silica | 0.09 |

Manufacturing Process

The various pulverulent components with the exception of the antistatic agent are mixed in a mixer/granulator of the ROTOLAB ZANCHETTA® mixer/granulator/drier under vacuum type, or equivalent, until the mixture is homogenized. A wetting solution or suspension comprising the liquid component(s) is then incorporated with stirring in order to obtain a wet granule.

This granule is then dried under suitable conditions in order to evaporate the granulation solvent, calibrated, and then micronized using an air jet micronization machine of the GALETTE ALPINE 200AS or JETMIL type (or equivalent).

Micronization Parameter:

Injector: 8 Bars, Crown: 6 Bars, Speed: 25 Kg/h

In order to reduce the agglomeration phenomenon due to the low particle size of the micronized powder, an antistatic agent (colloidal silica) previously screened is added by progressive mixing in a Turbula mixer.

Granule Control Before Micronization

Particle size: carried out by using a laser granulometer Malvern Mastersizer 2000 equipped with a vibrator Sirocco 2000
Parameters: Pression=2 bars; Vibration=80%
Result: Mean particle size=157.98 μm
Flowability: according to european pharmacopoeia test 4.2; 2.9.16
Flow
Sample mass=100 g, Flow time=∞
Apparent volume: according to european pharmacopoeia test 4.2; 2.9.15
Sample mass=100 g
Apparent volume at V0=166 mL
Apparent volume at V10=156 mL
Apparent volume at V500=148 mL
V10-V500=6 mL
Measurement of the relative humidity level: carried out by using a humidity analyser MA 30 Sartorius
Parameters: sample mass=2 g, Temperature=75° C.,
Dessication time=automatic
Result: Relative humidity=1.41%
   Control on Final Micronized Powder
Particle size: carried out by using a laser granulometer Malvern Mastersizer 2000 equipped with a vibrator Sirocco 2000
Parameters: Pression=3 bars; Vibration=70%
Result: Mean particle size=2.349 μm
Flowability: according to european pharmacopoeia test 4.2; 2.9.16
Flow
Sample mass=100 g, Flow time=∞
Apparent volume: according to european pharmacopoeia test 4.2; 2.9.15
Sample mass=50 g
Apparent volume at V0=178 mL
Apparent volume at V10=170 mL
Apparent volume at V500=164 mL
V10-V500=8 mL
Measurement of the relative humidity level: carried out by using a humidity analyser MA 30 Sartorius
Parameters: sample mass=about 3 g, Temperature=75° C., Dessication time=automatic, Test number=3
Result: Mean relative humidity=1.08%
Dissolution kinetic in vitro

Operating conditions: 1 g of micronized powder is dissolved at 37° C. in 10 g of medium, with magentic stirring at 500 RPM

TABLE 6

| Medium | Time (s) |
|---|---|
| Phosphate buffer pH 4.5 | 4.63 |
| Phosphate buffer pH 8 | 8.36 |
| Phosphate buffer pH 7.4 | 5.87 |
| Artifical saliva | 2.72 |

The grain size distribution profile of the powder according to example 2, before micronization, is illustrated in the FIG. 1.

### EXAMPLE 3

### Immediate-Release Powder According to the Invention

A Powder having the following weight composition is prepared:

TABLE 7

| Composition | Quantity in % |
|---|---|
| Testosterone | 10 |
| Dextran | 87.91 |
| Glycerol | 1.99 |
| Colloidal silica | 0.1 |

Manufacturing Process:

The various pulverulent components with the exception of the antistatic agent are mixed in a mixer-granulator of the mixer-granulator-fluidized bed dryer type equipped with a top spray nozzle or equivalent, until the mixture is homogenized. Then, a wetting solution or suspension comprising the liquid component(s) is sprayed using a spray nozzle on the product in motion in order to simultaneously distribute the solution homogenously and to dry it so as to evaporate the granulation solvent.

This granule is calibred, and then micronized with the aid of an air jet micronization equipment of the GALETTE ALPINE 200AS or JETMIL type (or equivalent). The setting parameters are identical to those described in the example 1.

In order to reduce the agglomeration phenomenon due to the low particle size of the micronized powder, an antistatic agent (colloidal silica) previously screened is added by progressive mixing in a Turbula mixer.

Control on Final Micronized Powder

Dissolution kinetic in vitro

Operating conditions: 1 g of micronized powders is dissolved at 37° C. in 10 g of medium, with magentic stirring at 500 RPM

TABLE 8

| Medium | Time (s) |
|---|---|
| Phosphate buffer pH 4.5 | 8.9 |
| Phosphate buffer pH 8 | 7.23 |
| Phosphate buffer pH 7.4 | 7.74 |
| Artifical saliva | 6.78 |

The grain size distribution profile of the powder according to example 3, before micronization, is illustrated in the FIG. 2.

### EXEMPLE 4

### Immediate-Release Powder According to the Invention

A Powder having the following weight composition is prepared:

TABLE 9

| Composition | Quantity in % |
|---|---|
| Dihydrotestosterone | 5 |
| Mannitol | 90 |
| Propylene glycol | 3 |

Manufacturing Process:

According to example 2

Control on Final Micronized Powder

Dissolution kinetic in vitro

Operating conditions: 1 g of micronized powder is dissolved at 37° C. in 10 g of medium, with magentic stirring at 500 RPM

TABLE 10

| Medium | Time (s) |
|---|---|
| Phosphate buffer pH 4.5 | 6.28 |
| Phosphate buffer pH 8 | 7.71 |
| Phosphate buffer pH 7.4 | 6.14 |
| Artifical saliva | 4.97 |

1. Immediate-release pharmaceutical or nutraceutical micronized powder having a particle size of at most 100 μm and comprising a combination of at least one active substance, at least one wetting agent and at least one diluent.

2. Powder according to claim 1, characterized in that it has a particle size of at most 50 μm.

3. Powder according to claim 1, characterized in that it has a particle size of at most 10 μm.

4. Powder according to claim 1, characterized in that it allows the dissolution of all of the active substance(s) in less than 30 seconds, when it is administered mucosally.

5. Powder according to claim 1, characterized in that the active substance is in a micronized form.

6. Powder according to claim 1, wherein the active substance is at least one member selected from the group consisting of cyproterone acetate, norethisterone acetate, progesterone, 3-keto-desogestrel, norgestimate, laevonorgestrel, desogestrel, gestodene, a natural estrogens a synthetic estrogen, Δ-4-androstenedione, testosterone, dihydrotestosterone, or androstanolone, DHEA, trinitrine, fentanyl, nitroglycerine, nicotine (nicotine S(−)), scopolamine, clonidine, isosorbide dinitrate, alclometasone dipropionate, phloroglucinol, molsidomine, acetazolamide, acyclovir, adapalene, alclomethasone dipropionate, amcinonide, ameline, bamethan sulphate+escin, betamethasone valerate, betamethasone dipropionate, bufexamac, caffeine, calcipotriol monohydrate, cetrimonium bromide, clobetasol propionate, crilanomer, desonide, dexpanthenol, diclofenac, diflucortolone, valerate, difluprednate, diphenydramine hydrochloride, econazole nitrate, erythromicin, flumetasone pivalate, fluocinolone acetonide, fluocinodine, fluocortolone, fluocortolone hexanoate, fluocortolone pivalate, hydrocortisone, hydrocortisone acetate, ibacitabine, ibuprofen, imiquimod, ketoconazole, ketoprofen, lidocaine, metronidazole, miconazole nitrate, minoxidil, nifluminic acid, penciclovir, benzoyl peroxide, piroxam, iodinated povidone, promestriene, pyrazinobutazone, roxithromycin, sulphacetamide, triamcinolone, tazarotene, tretinoin and isotretinoin, triclocarban, vidarabine monophosphate, β-3-adrenergic agonist, growth hormone, oxybutinin, buprenorphine, pergolide, nestorone, 7α-methyl-19-nortesterone, mecamylamine, salbutamol, clenbuterol, selegiline, buspirone, ketotifen, lidocaine, ketorolac, eptazocine, insulin, α-interferon, prostaglandins, 5-aminolevulinic acid, benzodiazepine alprozolam, diclofenac, fenoprofen, flubiprofen, ketoprofen, methyl phenidate, miconazole, piroxicam, bruprenorphine, alprozolam, dexmedetomidine, prazosin (α-adrenergic antagonist), alprostadil, tulobuterol (β-adrenergic agonist), ethinyl oestradiol+norelgestromin, ketorolac, physostigmine, medindolol (β-adrenergic agonist), rotigotine (dopamine D2 antagonist), thiatolserine, Esomeprazole, Melagatran (in the case of thrombosis), Rosuvastatin, Ezetimide, Pitavastatin (hyperlipidaemia), Mitiglinide (type II diabetes), Cilomilast, Viozan (asthma), Aripipazole (psychiatry), Omapatrilat (hypertensive), Orzel (cancerology), Caspofongin acetate, Voriconazole (infections), a new COX inhibitor, Valdecoxib (arthritis) and Parecoxib, Substance P antagonist (depression), Darifenacin (urology), Eletriptan (migraine), Alosetron, Tegaserod, Capravirine (HIV), Finasteride (5-alpha reductase inhibitor).

7. Powder according to claim 1 wherein the active substance is at least one member selected from the group consisting of a vitamin, an inorganic salt, and brewer's yeast.

8. Powder according to claim 1, wherein wetting agent is at lease one member selected from the group consisting of a polyol, triacetin, a hydrogenated vegetable oil, a polyoxy(ethylene)polyoxy(propylene) copolymer, and a polyoxyethylene alkyl ether.

9. Powder according to claim 1, wherein the diluent is at least one member selected from the group consisting of calcium or sodium carbonate or bicarbonate, sucrose, mannitol, xylitol, sorbitol, lactose, maltotol, glucose, cellulose, a microcrystalline cellulose powder, starch, a starch derivative, dibasic calcium phosphate, tribasic calcium phosphate, calcium sulphate, a dextrate, a dextrin, a dextrose excipient, fructose, kaolin, and lactitol.

10. Powder according to claim 1, characterized in that it further comprises an antistatic agent.

11. Powder according to claim 10, characterized in that the antistatic agent is selected from the group consisting of colloidal silica, magnesium silicate, talc, calcium silicate and tribasic calcium phosphate and mixtures thereof.

12. Powder according to claim 1, which further comprises a binder which is at least one member selected from the group consisting of acacia, alginic acid, carboxymethyl cellulose sodium, microcrystalline cellulose, a dextrin, ethyl cellulose, gelatin, glucose, guar gum, hydroxypropyl methyl cellulose, methyl cellulose, polyethylene oxide, povidone, and pregelatinized starch.

13. Powder according to claim 1, which further comprises an absorption enhancer selected from the group consisting of an aliphatic fatty acid ester, a fatty acid, an alcohol or polyol, a component of an essential oil, a terpene derivative, a surfactant, a moisturizer, a keratolytic agent, 23-lauryl ether, aprotinin, azone, benzalkonium chloride, cetylpyridinium chloride, cetyltrimethylammonium bromide, a cyclodextrin, dextran sulphate, lauric acid, lysophosphatidylcholine, a menthol, methoxysalicylate, methyl oleate, oleic acid, phosphatidylcholine, polyoxyethylene, polysorbate 80, sodium EDTA, sodium glycocholate, sodium glycodeoxycholate, sodium lauryl sulphate, sodium salicylate, sodium taurocholate, sodium taurodeoxycholate, a sulphoxides, and an alkyl glycoside.

14. Powder according to claim 1, characterized in that it further comprises an edulcorant agent and/or a flavoring agent.

15. Powder according to claim 14, characterized in that the edulcorant agent is selected from the group consisting of aspartam, dextrates, dextrose, fructose, mannitol, sodium or calcium saccharinate, sorbitol, sucralose, sucrose, and mixtures thereof.

16. Powder according to claim 14, wherein the flavoring agent is at least one member selected from the group consisting of a flavor of synthetic, semi-synthetic or natural origin.

17. Powder according to claim 1, characterized in that it is in a form suitable for its application on the buccal mucosa, the nasal mucosa or the vaginal mucosa.

18. Powder according to claim 1 characterized in that it is in a form suitable for its application to the buccal mucosa sublingually.

19. Powder according to claim 1, characterized in that it is in a sprayable form.

20. Powder according to claim 1, characterized in that it is packaged in a single-dose packet.

21. Powder according to claim 1, characterized in that it is packaged in a thermally moulded capsule provided with a peelable operculum.

22. Powder according to claim 1, characterized in that it is in a packaging suitable for powder administration known to those skilled in the art.

23. (canceled)

24. Powder according to claim 6 wherein the active substance is at least one member selected from the group consisting of estradiol, a natural estradiol derivative, ethinylestradiol, and Etoricoxib (inflammation).

25. Powder according to claim 8 wherein the wetting agent is at least one member selected from the group consisting of PEG, hexylene glycol, triacetin, and a hydrogenated vegetable oil.

26. Powder according to claim 13 wherein the absorption enhancer is at least one member selected from the group consisting of isopropylmyristate, oleic acid, ethanol, propylene glycol, polyethylene glycol, eugenol, geraniol, nerol, eucalyptol, menthol, polyoxyethylene sorbitan, polyoxyethylene alkyl ether, polyoxyethylene derived from castor oil, glycerin, urea, and an alpha-hydroxy acid.

27. A method for manufacturing an immediate-release pharmaceutical or nutraceutical composition, said method comprising a step of micronizing a composition comprising a combination of at least one active substance, at least one wetting agent and at least one diluent.
